(19) United States
(12) Patent Application Publication
(10) Pub. No.: US 2005/0256067 A1
(43) Pub. Date: Nov. 17, 2005

(54) Method of preparing a treatment product, treatment product and a plasmid construct

(76) Inventors: Jarmo Wahlfors, Kaupio (FI); Tiina Wahlfors, Kuopio (FI)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/505,933

(22) PCT Filed: Feb. 27, 2003
(86) PCT No.: PCT/FI03/00138

§ 371 Date: Jun. 13, 2005

(30) Foreign Application Priority Data
Feb. 27, 2002 (FI) 20020375

(51) Int. Cl. A61K 48/00

(52) U.S. Cl. 514/44; 424/93.2

(57) ABSTRACT

The method of the invention for preparing a treatment product is characterized by using a starting plasmid based on a virus belonging to the Togaviridae stock from which the genes encoding capsid proteins of the virus have been deleted. An RNA encoding virus-like particles (VLP-RNA) is prepared by manipulating the staring plasmid by connecting to it a spreading enabling gene and a treatment gene. The invention is furthermore concerned with such a treatment product and a plasmid construct encoding virus-like particles, which is prepared from the Sindbis virus in which the capsid protein of the virus has been substituted by a spreading enabling gene and a treatment gene. Example of spreading enabling gene is a gene encoding vesicular stomatitis virus glycoprotein (VSV-G). A particular example of treatment product is Herpes simplex virus thymidine kinase linked to GFP, said product being Spicude/Reporter construct

### TECHNICAL FIELD

The invention is concerned with a method of preparing a treatment product, a treatment product and a plasmid construct. The product is intended for forming virus-like particles (VLP) and is used for treatment of cancer and as a vaccine.

### TECHNICAL BACKGROUND

The DNA of cells of human beings and animals often contains genetic errors causing the cell to divide in an uncontrolled way or other diseases. In cancer it is question about cells, which replicate in an uncontrolled way because of an erroneous DNA function.

In gene therapy, the goal is to substitute sick genes with healthy genes or to transfer such genes into the cells, which destroy malicious cells. So called gene vehicles are needed for the transfer of new genes to a target cell.

Gene therapy is one of the most important domains of study in modern biomedicine. In recent years, extensive attempts have been made especially for the investigation of gene therapeutic treatment of cancer. The development and characterization of proper and efficient gene vehicles belong to the most important challenges of the branch. Gene treatment requires very efficient methods to work for the transport of the genes and on the other hand also for the expression of the transfer genes in the target, i.e. cancer, tissue.

The most common genetic vehicles in the investigations of gene therapy of cancer have been retrovirus and adenovirus based vehicles and the liposomes. The gene transfer efficiency of the liposomes has, however, been very low compared to the effect of viral vehicles even of the safety questions can be eliminated by using liposome based vehicles. Thus, virus based gene transfer methods have in practice been the only methods efficient enough for gene therapy of cancer.

Alphavirus based gene vehicles have lately been used for more and more applications. Typical for alphavirus vehicles is that they can infect the target cell relatively efficiently and the expression of the transfer gene to be transport in those in the target tissue is powerful and has a short duration.

Characteristic for alphaviruses (especially Sindbis and Semliki forest viruses) is a very quick and efficient replication inside the host cell.

The Sindbis virus is a typical alphavirus. In addition to Sindbis, 26 other viruses belong to the alphaviruses, which all have a very similar structure. The alphaviruses are considered to belong to arbo viruses and they belong to the Togaviridae stocks. The plus stranded RNA genome in the alphaviruses are surrounded by the shell i.e. the capsid is formed by the own proteins of the virus. The capsid and the RNA genome together= nucleocapsid. Around the capsid there are furthermore cellular membrane formations with the origin in the host cell of the virus to which formations own proteins of the virus are connected. This construction is called a coat. When the alphavirus adheres to its target cell, its coat is fused into the cell membrane of the target cell and the nucleocapsid is released to the cell. Thereafter the capsid disintegrates and the RNA genome is released to the cell slim and starts to replicate.

Natural wide-type viruses can be converted to so called recombinant systems, with which a controlled gene transfer is achieved. Alphavirus based recombinant systems are based on the fact that the sequence encoding the capsid proteins of the virus (the subgenomic RNA) can be deleted and substituted by foreign genetic material. In this case, there is usually first prepared a plasmid from the alphavirus by isolating the virus genome and by copying the virus RNA to an annular DNA structure, i.e. a plasmid. This plasmid can be further manipulated by deleting the structural genes encoding the is capsid proteins of the virus and by substituting them by the desired transfer genes. RNA can be synthesized in a test tube (in vitro) by means of the plasmid and by means of this, a recombinant virus can be produced, which can not spread in the target cell system.

In this way, a situation is achieved, wherein the virus can not replicated anymore in an uncontrolled way (and cause infections), and so a big amount of foreign genetic products is produced instead of virus capsids. Recombinant forms of alphaviruses have earlier primary been used for the production of proteins in animal cells, but lately they have been used as gene vehicles as well. When alphavirus based recombinant systems are used as gene vehicles, also the genetic code of the virus capsids is necessary to achieve virus particles. The missing genetic code of the virus capsids therefor has to be fetched externally (in trans) by means of a so called helper RNA.

Thus, alphavirus vehicles are normally produced by simultaneously transferring a vector RNA to the producer cells by means of electroporation, which vector RNA has the desired gene instead of the structural gene, and a helper RNA, which as a separate unit brings the code for the structural protein. A big amount of virus particles are formed outside the cells as a result of a co-operation of these RNA molecules, which virus particles have the ability to infect many different types of target cells. The U.S. Pat. No. 5,843,723 as well as WO 94/17813 and WO 96/37616 are mentioned as prior art.

In this connection, the words vector and replicon are used in such a way that by vector there is generally meant a vehicle, in other words any system (a lipid sphere, a gold particle, a virus) with which genetic material can be brought to the target cells. In this connection, the vector is also called “gene transfer vector”. A replicon again is at least in this connection an RNA molecule, which contains instructions for its duplication, i.e. replication.

Thus, forming of virus capsids or virions are needed for the production of viruses and virus vectors. The virions have a regular structure, which consists of a so called capsid formed by the own protein of the virus, and of a coat with the origin in the membrane of the host cell, which furthermore has the own coat protein of the protein integrated. In given conditions, for example through mutation, it is, however, possible that particles are formed, which lacks a normal, regular virus structure. This are called virus-like particles, VLP, or pseudoviruses.

Rolls and his colleagues (Rolls et al, 1994, Cell 79, 497-506) observed that VL particles also can be formed if a coat protein from an other virus (VSV-G, vesicular stomatitis glykoprotein virus) is connected to the subgenomic sequence encoding the structural proteins of an alphavirus, the semliki forest virus. These VL particles are infectious thanks to the VSV-G coat protein. They called these VL particles with the name “minimal RNA virus”. The particles were shown to be of membrane particles that were released from the host cell and have VSV-G protein on their surface. The membrane particles can fuse with the neighbor cells by means of the surface protein and if they have a VLP-RNA molecule (an RNA encoding VIP) inside them, its replication and spreading starts again.

Later, the same group showed that by connecting some other gene beside the VSV-G gene of the RNA molecule, also the expression of this gene can be spread (Rolls et al, 1996, Virology, 218, 406-411). This working group showed in the middle of the last decade that an infected particle can be much simpler than viruses in general. The observation has, however, not been made use of and the possibilities to use these “minimal RNA viruses”(in other words virus like particles, VLP) have not been investigated or reported in the literature.

### THE OBJECT OF THE INVENTION

Improvement of the efficiency and safety of gene transfers is a continuous goal in the branch and the object of the invention is to develop better gene transfer methods and products and their possibilities of use.

### SUMMARY OF THE INVENTION

The method of the invention for preparing a treatment product is characterized by using a starting plasmid based on a virus belonging to the Togaviridae stock from which the genes encoding capsid proteins of the virus have been deleted. An RNA encoding virus-like particles (VLP-RNA) is prepared by manipulating the starting plasmid by connecting to it a spreading enabling gene and a treatment gene.

The starting plasmid can be based on an alphavirus, especially the Sindbis or Semliki forest virus, whereby it for example is the pSFVdpG-X plasmid of the Semliki forest virus or the pSinRep5 plasmid of the Sindbis virus.

The invention is furthermore concerned with a treatment product, which is characterized by bat it contains an RNA encoding virus like particles (VLP-RNA), that contains a spreading enabling gene and a treatment gene. Still further, the invention is concerned with a plasmid construct encoding virus-like particles, which is characterized by that it is a construct prepared from the Sindbis virus in which the capsid protein of the virus has been substituted by a spreading enabling gene and a treatment gene.

Preferable embodiments of the invention have the characteristics of the subclaims, and are presented in the following.

The pSFV-G-dp-TKGFP-plasmid construct respective the pSin-G-dp-TKGFP-plasmid construct are prepared from the starting plasmids.

The pSFV-G-dp-TKGFP-plasmid construct is prepared by opening the pSFVdpG-X-plasmid by a restriction enzyme and by connecting the transfer gene TKGFP to the opened plasmid, whereas the pSin-G-TKGFP-plasmid construct is prepared by opening the pSinRep5-plasmid with a restriction enzyme arm by connecting VSV-G and TKGFP to the opened plasmid.

More in detail, the pSin-G-dp-TKGFP-plasmid is prepared from the pSinRep5 plasmid by connecting the VSV-G gene to the plasmid; by connecting a second subgenomic promoter to the plasmid, by connecting the TKGFP gene after the foregoing one, by isolating the pSin-G-dp-TKGFP-plasmid and by mass producing the plasmid for RNA-synthesis.

The prepared VLP-RNA are brought to the target cells by connecting it to bearer particles, which are microscopic gold particles coated with VLP-RNA and then shot to the cells by means of a helium gas impulse.

Alternatively, the VLP-RNA is brought to the target cells by electroporation, by direct injection or by transfection.

The plasmid construct of the invention has preferably two subgenomic units, one of which encodes the VSV-G protein and the other one a transfer gene, whereby the transfer gene is e.g. the fusion TKGFP of a treatment gene and a marker gene. pSin-G-dp-TKGFP is such a plasmid construct.

The method of the invention can be used for gene therapy of cancer. Hereby, VLP-RNA is synthesized by using, as a transfer gene a gene that is toxic for the cancer cell and/or a gene that improves the immune response. The synthesized RNA is fastened to the bearer particles and the resulting particles are shot to the tumor by means of a gas impulse in order to form VL particles in the cancer cell. The transfer gene used in this use is for example the thymidine kinase gene HSV-TK or herpes simplex virus type 1.

When the method of the invention is used as a vaccine, the VLP-RNA is synthesized by using a vaccine gene as a transfer gene and/or a gene that improves the immune response. As in the cancer therapy, the RNA is fastened to bearer particles and the resulting particles are shot to the skin by means of a gas impulse in order to form VL particles in the skin. In this use, the transfer gene is the surface protein gene of HIV-1 virus p21, for example the interleukin-12 gene from a gene that increases the immune response.

A still further use of the method of the invention is the preventing of the re-growth of blood vessels. In this case, the VLP-RNA is synthesized by using as a transfer gene a gene that prevents the growth of the cells. The RNA is fastened to the gene transfer material and the RNA-bearer mixture is injected to the inner wall of the blood vessel in a connection with balloon extension in order to form VL particles in the cells in the inner wall of the blood vessel. The transfer gene in this use is for example HSV-TK.

Thus, by the method of the invention, instead of conventional virus vehicles, so called virus-like particles (VLP) can be produced for gene transfers which lacks a normal capsid construction of the virus. These particles are not formed until in the target tissue, to which RNA molecules that encode these are brought for example by shooting by means of bearer particles (the so called gene gun method). The formed VL particles furthermore have such properties that after their forming they can be spread to neighboring cells and form new particles. In such a way a considerable improvement of the gene transfer efficiency can be achieved in a cancer issue and thus a successful treatment is enabled (a low gene transfer efficiency is for time being the worst problem in the development of gene therapies of cancer).

In the method, alphaviruses are used for the production of virus-like particulars (VLP), which are replicated very efficiently in many cancer cells. The biological background of the method is complicated and it has been presented more in detailed in the detailed part of the invention. Simply, the functioning of the or some embodiments of the method is the following:
VL particles encoding RNA is prepared by manipulating alphavirus-RNA by deleting the own genes of the virus from it and by substituting them with 1. a gene that prevents spreading and 2. a treatment gene for cancer. The manipulation takes place by first preparing a starting plasmid from the alphavirus that has a genetic code for the vector needed. The starting plasmid can for example be either one from the article (“Expression of Additional Genes in Vector Derived from a Minimal RNA virus”, Rolls et al 1996, Virology 218, 406-411 known as plasmid pSFVdpG-X or the pSINRep5-plasmid achieved from the company Invitrogen. The first mentioned plasmid is prepared from the Semiliki forest virus (SFV) and the second one from the Sindbis virus.

The starting plasmids are further manipulated in order to obtain a pSFV-G-dp-TKGFP resp. a pSIN-G-dp-TKGFP-plasmid construct for example when the intention is gene therapy of cancer. In the other uses of the invention other transfer genes are used in order to obtain plasmid constructs to be further manipulated from which virus-like particles encoding VLP-RNA can be prepared. Both the above mentioned plasmids contain the TKGFP-treatment gene instead of the capsidl protein of the virus. Furthermore, pSIN-G-dp-TKGFP is an object of the invention.
A VLP-RNA vector is synthetized from these plasmids e.g. in a test tube and is purified.
The VLP-RNA is fastened to the surface of bearer particles in order to transport the particles to the target cell.
The particles are transported to the cancer tissue, whereby RNA is released to the cells.
The RNA starts its replication and simultaneously VL particles are formed from the cells.
The particles are released from the cells and they transport RNA molecules inside themselves which again can replicate when reaching the neighbor cells and form new particles.

In this way a controlled infection has been achieved only by transferring pure RNA, thanks to which the real treatment gene for the cancer spreads all over the tumor. The infection does not spread outside the cancer tissue as the forming of the VL particles require dividing of the cells (which in practice only takes place in cancer cells).

In the invention, a replicon based on the alphavirus genome is produced from the above plasmids, which can produce virus-like particles when transported to the cells. In the replicon in question, all structural protein genes of the alphavirus have been substituted with a glycoprotein encoding gene (VSV-G) of the coat with the origin in another virus (vesicular stomatitis virus, VSV), which makes the replicon infective. Thanks to the expressing VSV-G glycoprotein, the replicon can spread in the virus-like particles, which are released by means of VSV-G from the cell membrane of the host cell infected by the replicon.

The Sindbis virus based VIP, wherein the plasmid of the invention has been used, works more efficiently than the SFV-based as more VL particles are formed and they spread to more neighboring cells.

The invention also provides the following benefits:
The infection is restricted only to dividing cells, i.e. cancer cells, and no healthy tissue is infected or destroyed. This is because the spreading of the particles require cancer cell like active cell division. In other words, if the growth of the cells stops as in normal cells, then the VLP infection does not proceed either. This is a very positive feature in view of the usefulness of the method.
The method of the invention can be used in several different target cell lines. In tests, the spreading has been observed in e.g. the BHK-21 target cell line, where the VLP infection achieved with the method of the invention works very well and in two brain tumor cell lines, wherein the spreading proceeds slower though
The VLP-RNA can be transported to the target cells connected to bearer particles. In this so called gene gun method, the microscopic gold particulars are coated with VLP-RNA and are shot to the cells by means of a helium gas impulse. In the BHK cells, the infection starts efficiently in those cells to which RNA has been shot Thereafter, the fluorescence and cell death are transferred to the neighboring cells as long as the cells are divided.

In accordance with the invention, it is thus clear that alphavirus based VL particles can be used as gene vehicles and the production can be started in the target tissue by means of pure RNA (without any production or purifying of the viruses).

The method of the invention can be used for gene therapy of cancer, in vaccine use or for preventing re-growth of blood vessels. The treatment gene is in this case herpes simplex virus type 1 thymidine kinase gene (HSV-TK), the surface protein gene of HIV-1 virus p21 and the interleukin-12 gene.

In the following, the invention is described for illustrating purposes by means of figures and examples, which are not meant to restrict the invention. In the invention, for example the treatment gene can be another gene than the presented one and the details of the method can naturally differ from what has been presented in the following in the scope of the knowledge of one skilled in the art.

### FIGURES

FIG. 1 presents the pSFVdpG-X- starting plasmid.

FIG. 2 presents the pSFV-G-dp-TKGFP plasmid construct.

FIG. 3 presents the pSINREP5 starting plasmid.

FIG. 4 presents the pSin-G-dp-TKGFP plasmid of the invention.

FIG. 5 presents the replication cycle of alphaviruses generally.

FIG. 6 preparation the principle of the preparation of the alphavirus vectors of prior art.

FIG. 7 presents a first use of the invention for gene therapy of cancer.

FIG. 8 presents a second use of the invention for vaccine use.

FIG. 9 presents a third use of the invention for preventing re-growth of blood vessels.

FIG. 10 presents the VLP-RNA of the plasmids of FIGS. 2 and 4 and the expression of the comparison RNA in BHK cells.

FIG. 10b presents the expression of the VLP-RNA of the plasmid of FIG. 4 in the BHK cells in different cell densities.

FIG. 11a presents the expression of the VLP-RNA of the plasmid of FIG. 2 in MBA13 cells.

FIG. 11b presents the expression of the VLP-RNA of the plasmid of FIG. 4 in MBA13 cells.

FIG. 12a presents the function of the VLP-RNA of the plasmid of FIG. 2 in a gene therapy test in MBA13 cells.

FIG. 13 presents the transfer of the RNA of the invention to BHK cells by means of the gene gun method and its functionality.

### DETAILED DESCRIPTION OF THE INVENTION

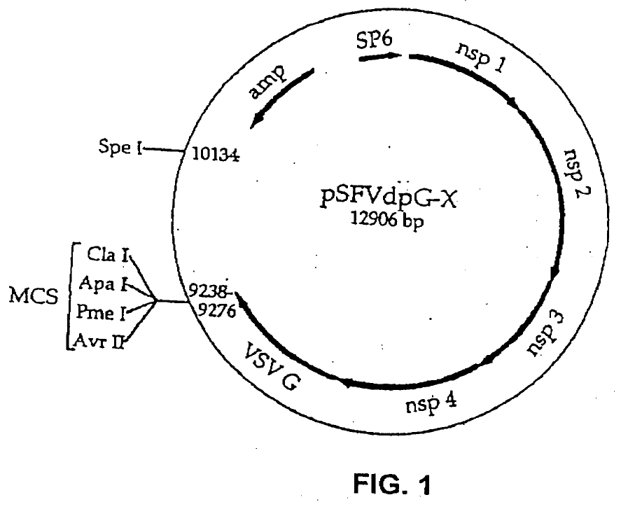

In the followings the method of the invention is described as an example by means of FIGS. 1-4 in other words how VLP-RNA can be prepared. In the description there has been described the preparation of a VLP-RNA intended for the treatment of cancer as an example. For other treatments, other transfer genes are used, such as TKGFP.

FIG. 1 presents the starting plasmid pSFVdpG-X prepared from the Semliki forest virus, herein the sequence that encodes the capsid protein of the vines has been deleted. The starting plasmid is in accordance with the article “Rolls et al (1996) Virology 218, 406-411”. The starting plasmid has, as a copy of the virus RNA, an annular DNA molecule with information for the replication of the virus. To this starting plasmid there has already been connected a VSV-G gene that enables the spading of the virus. SP6 prom. is a promoter sequence of the RNA polymerase enzyme in question. RNA is synthesized from the plasmids starting from this promotor sequence. nSP1-4 are so called non-structural protein genes of the virus. The products of these genes form a replicase-enzyme complex that enables the replication of the construct and the synthesis of the subgenomic RNA species (here VSV-G). A group of restriction places of the restriction endonucleases (BamHI, ClaI, PmeI. SpeI and PvuI) are indicated on the outer periphery of the plasmid figure, which are important for the further manipulation and characterization of the construct.

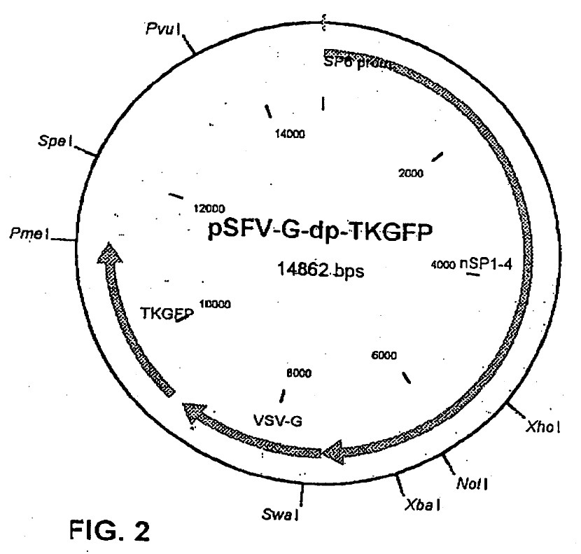

FIG. 2 presents the pSFV-G-dp-TKGFP-plasmid, which is prepared from the starting plasmid of FIG. 1. Hereby, the starting plasmid pSFVdpG-X of FIG. 1 is opened with the Pme 1 restriction enzyme and a transfer gene TKGFP is connected to the opened plasmid, which transfer gene is a fusion gene of the thymidine kinase gene (HSV-TK) of herpes simplex virus type 1 and the green fluorescent protein, GFP. The preparation and properties of this gene has been described in the publication “Loimas S, Wahlfors J, Janne J. (1998) Herpes simplex virus thymidine kinase—green fluorescent protein fusion gene: new tool for gene transfer studies and gene therapy Biotechniques 24 (4):614-8”. This transfer gene is used because by means of that both the gene transfer efficiency can be defined (GFP fluoresces with green color in UV-light, the positive cells can be observed by fluorescence microscope or by flow cytometry) and it works as a treatment gene (HSV-TK charges the virus medicine ganciclovir, GCV, in a toxic form, in other words the positive cells can be destroyed by means of GCV).

The resulting pSFV-G-dp-TKGFP plasmid of FIG. 2 is then isolated and the right structure is ensured by restriction enzyme analysis and the plasmid is mass grown for VLP-RNA synthesis. The meanings of the abbreviations presented in this figure are the same as in FIG. 1.

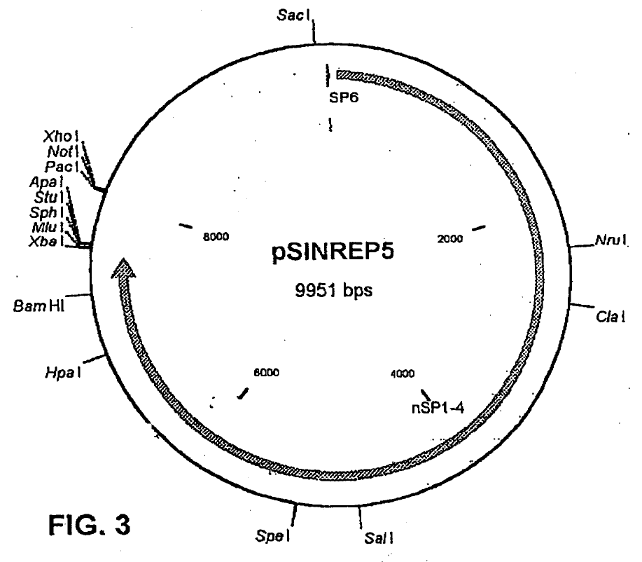

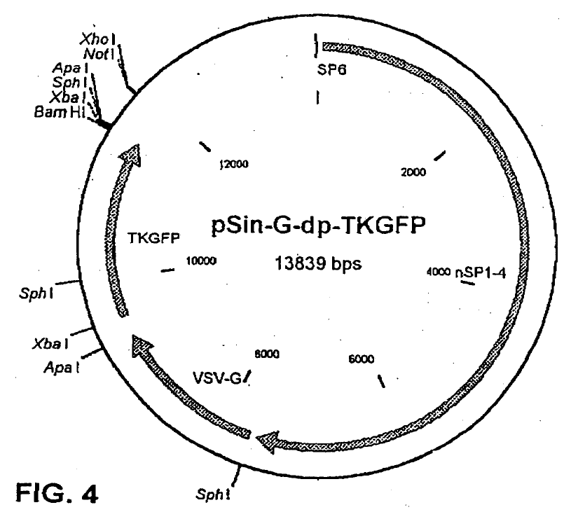

FIG. 4 presents the pSin-G-dp-TKGFP plasmid, which is achieved as a result when the pSinRep5 plasmid of FIG. 3 prepared from the Sinbis virus is manipulated.

Like the plasmid of FIG. 1, also the pSinRep5 plasmid of FIG. 3 is, as a copy of the virus RNA, an annular DNA molecule with information for the replication of the virus. The meanings of the abbreviations presented in this figure are the same as in FIG. 1.

In the starting plasmid of FIG. 3, the sequence encoding the capsid protein has been deleted and in FIG. 4 it has been substituted with an other gene, i.e. TKGFP transfer gene. In this figure, the meanings of the presented abbreviations are the same as in FIG. 1.

The pSinRep5 plasmid is achieved for the preparation of the pSin-G-dp-TKGFP plasmid for example from the Invitrogen company and it is opened with the Stu 1 restriction enzyme. The glycoprotein gene of vesicular stomatitis virus (VSV-G) is connected to the opened plasmid, which is released from the plasmid pMD.G. This plasmid has earlier been described in the article Naldini et al. 1996, “in vivo gene delivery and stable transduction of nondividing cells by a lentiviral vector” Science. 1996 Apr. 12; 272(5259):263-7. The pSin-VSV-G-plasmide is achieved as a result. This plasmid is further changed by deleing the region Sph I-Xba I which is harmful for the further operations with enzyme treatment between the restriction places.

The subgenomic promoter region is isolated from the pSinRep5 plasmid by using PCR-technique. By means of the primers 5′TT GGG CCC GGC GTG GTT TAG AGT AGG TA-3′ and 5′-GTA AGC GGG GAT CTA ATG-3′, a peace of ca 300 base pairs is replicated to which is first connected the pBLUESCRIPT plasmid (from the Stratagene company). The primer is a short one-stranded DNA piece, which is fastened to corresponding sequence according to normal pair forming rules (A→T 5 and C→G) e.g. in chromosomal DNA or, as here, in plasmid DNA. The primers are used to start the enzymatic reaction to replicate the given DNA region (= polymerase chain reaction, i.e. PCR). After checkings, this piece is manipulated by Apa I enzyme and connected to the plasmid pSIN-VSV-G at Apa I. The resulting plasmid is pSIN-G-dp1.

Plasmid pSIN-G-dp1 is opened with the Xba I restriction enzyme and the transfer gene TKGFP is connected to the opened plasmid. The pSIN-G-dp-TKGFP plasmid is isolated, the right structure is ensured by enzyme analysis and the plasmid is mass grown for RNA synthesis.

The synthesis of RNA from the above plasmids pSIN-G-TKGFP and pSFV-G-dp-TKFGP is performed in the invention e.g. so that the pSIN-G-dp-TKGFP plasmid is linearized with the Not I restriction enzyme and the pSFV-G-dp-TKGFP with the Spe I restriction enzyme. The linearized plasmid DNA is purified by phenol extraction and ethanol precipitation. One microgram of purified template-DNA is used for the RNA synthesis, which is performed by using SP6 mESSAGE mACHINE junction compound (Ambion). The produced RNA is precipitated by lithium chloride and diluted in water.

As was stated in the beginning of the application, it is generally characteristic for the alphaviruses that they replicate quickly and efficiently in the host cells.

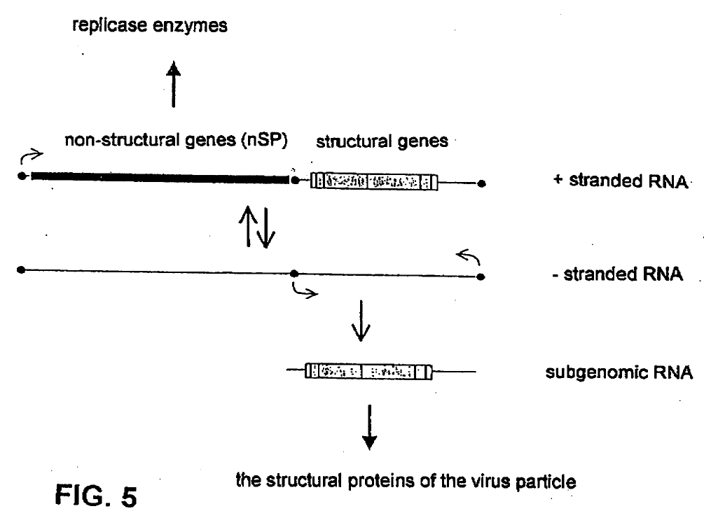

As a background, for an easier understanding of the invention, the replication cycle of alphaviruses has been presented in FIG. 5. In this cycle, there is first produced a to replication enzyme from the plus-stranded RNA of the virus, which in turn replicates 0 RNA through a minus-stranded intermediate. Furthermore, subgenomic RNA is produced from the minus-stranded RNA, which encodes structural proteins of the virus, in other words contains instructions for the production of virus particles.

In FIG. 5 it has been assumed that the alphavirus has adhered to the target cell. As a consequence of this, the virus releases plus-stranded RNA to the cell, which contains two different genomes, in other words a region that encodes structural genes and an area that encodes non-structural genes (nSP). Thus, non-structural genes are translated from the plus-stranded RNA, which encode an enzyme complex that causes the replication. The structural genes in turn encode the capsid proteins of the virus. Thus, them is first produced a replicas complex from the non-structural genes, which complex produces minus-stranded full length RNA. Subgenomic RNA is produced from the minus-stranded RNA, which subgenomic RNA encodes the structural proteins of the virus, in other words contains instructions for the production of virus particles. As plus-stranded RNA is achieved form the minus stranded RNA in the above described way, the replication of the RNA continues. When a sufficient amount of full-length, plus-stranded RNA has been formed, the process is stopped. The replicase-enzyme complex produces subgenomic RNA, i.e. structural protein. Then virus capsids are formed on the cell membrane on which plus stranded RNA is packaged. The replicase complex produces subgenomic RNA, i.e. structural protein. Virus capsids are then placed on the cell membrane on which plus stranded RNA is packaged.

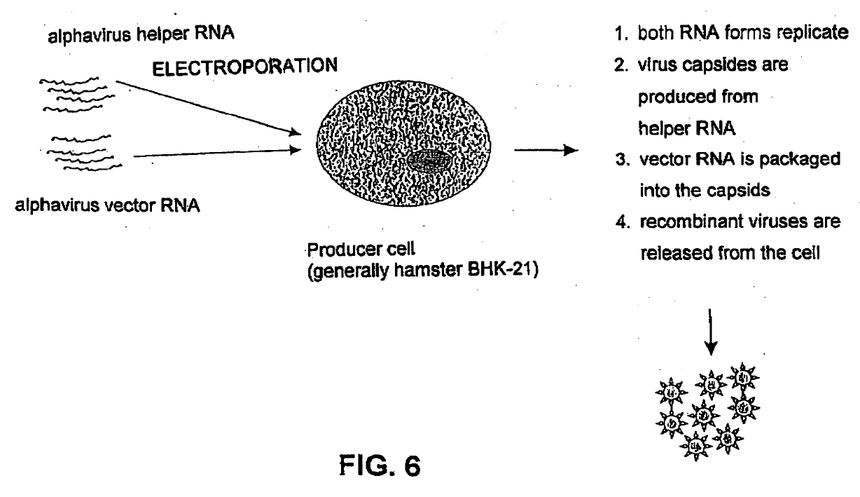

In FIG. 6 there has been presented for comparison, the principle of the production of alphavirus vectors of prior art. Thus, alpha virus helper RNA and alpha virus vector RNA are transported with electroporation to the producer cell, Which in general is the BHK-21 cell line of a hamster. As a consequence, both RNA formes are replicated in accordance with the principle presented in FIG. 5, i.e. virus capsids are produced from the helper RNA and vector RNA is packaged in the capsids. The recombinant viruses are thereafter released from the cells.

In the invention, contrary to what is presented in FIG. 6, there is first prepared a virus-like particles encoding RNA, i.e. from the plasmids pSFV-G-dp-TKFGP or pSIN-G-dp-TKGFP or from some other plasmid and then this VLP-RNA is transported to the target tissue. There, the RNA is let to replicate in order to form virus-like particles in the cells. The particles are released from the cells and infect the surrounding cells, hereby the RNA molecules transported inside them are replicated again and form new virus-like particles. As was stated earlier, the forming of virus capsids is normally needed for the production of viruses and virus vehicles, which in FIG. 6 is brought by means of helper RNA. The forming of the gene transfer vector and the controlled infection is achieved in the invention by means of pure vector RNA. The infection starts by means of the replication of FIG. 5 and is based on the forming of virus-like particles instead of normal virus particles.

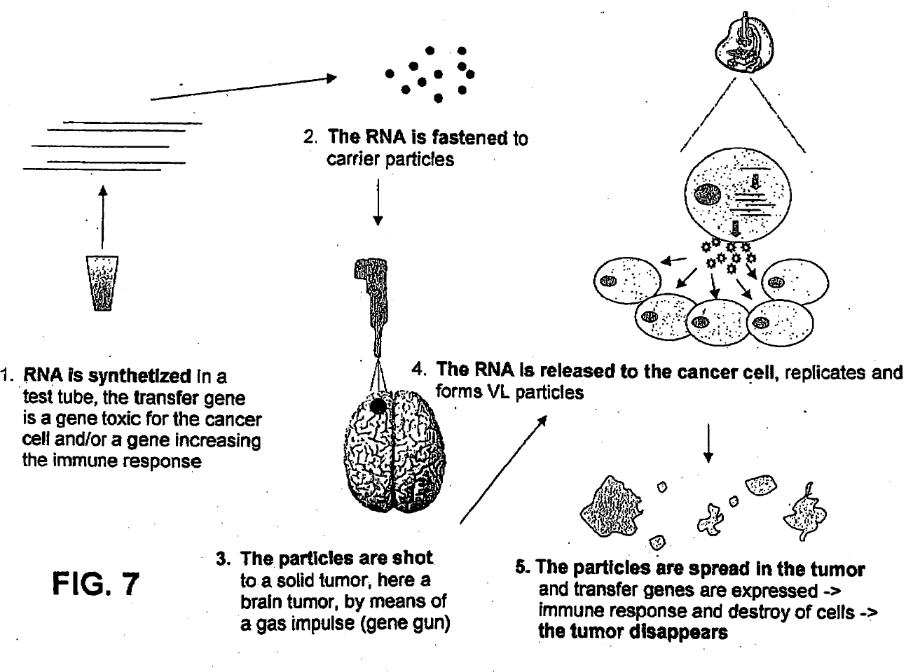

In FIGS. 7-9 different uses of the method of the invention have been presented.

In FIG. 7, there has been presented a first use of the invention implemented for gene they of cancer. In step 1, RNA is synthesized in a test tube as is described in connection with FIGS. 1-4. The transfer gene used is a gene that is toxic for the cancer cells and/or a gene that increases the immune response, for example the thymidine kinase gene HSV-TK of herpes simplex virus type 1 (the product of this gene in other words changes the harmless virus medicine ganciclovir in a toxic form). In step 2, the synthesized RNA is fastened to bearer particles. In step 3, these particles are shot in the solid tumor, which here is a brain tumor by means of a gas impulse (the so called gene gun method). In step 4, the RNA is released to the cancer cell, is replicated in accordance with FIG. 5 and forms VL particles. In step 5, the particles are spread in the tumor and the transfer genes are expressed. The result is an immune response and the destruction of cells as a consequence of which the tumor disappears.

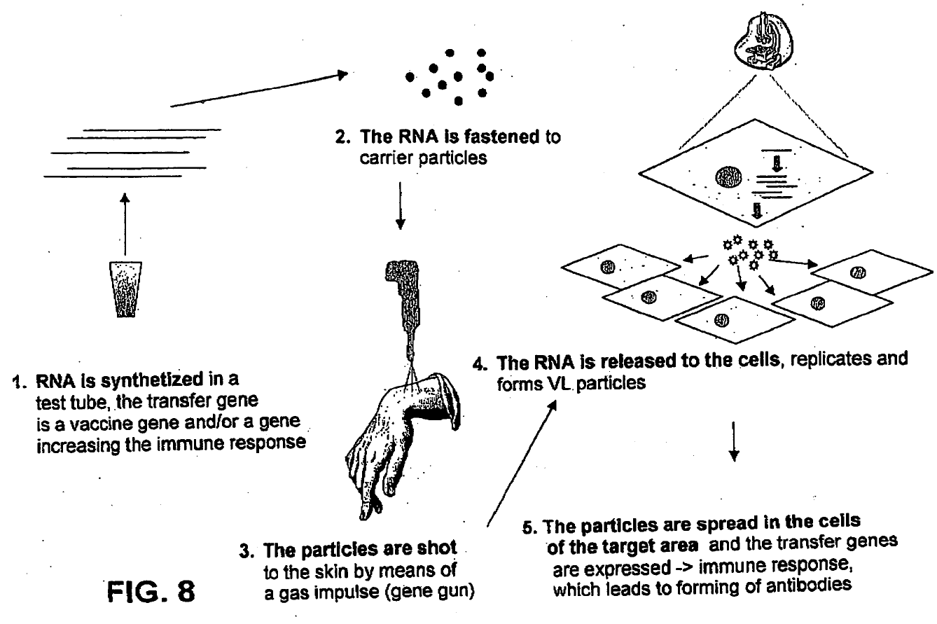

In FIG. 8, there has been presented a second use of the invention implemented for vaccine use. In step 1, RNA is synthesized in a test tube. The transfer gene used is a vaccine gene and/or a gene that increases the immune response, for example the surface protein gene of HIV-1 p21 as a vaccine gene, or interleukin-12 gene as a gene that increases the immune response. In step 2 the RNA is fastened to bearer particles. In step 3, the particles are shot to the skin cells by means of a gas impulse (with a gene gun method). In step 4, the RNA is released to the cells of the skin, is replicated in accordance FIG. 5 and forms VL particles. In step 5, the particles are spread to the cells of the target area and the transfer genes are expressed. An immune reaction is formed, which leads to the forming of antibodies and/or a cell mediated immunity.

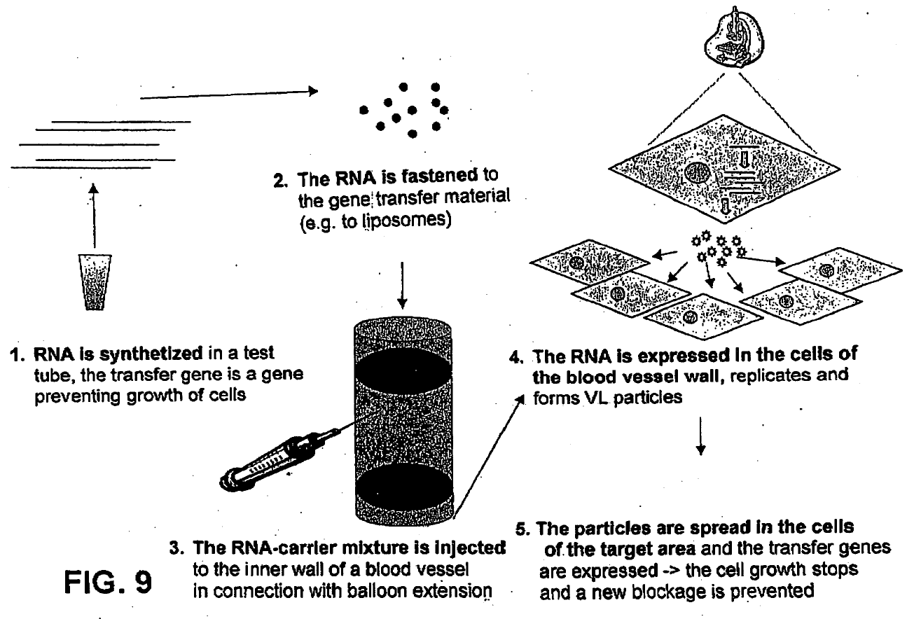

In FIG. 9, there has been presented a third use of the invention for preventing re-growth of blood vessels. In step 1, RNA is synthetized in a test tube. The transfer gene used is a gene that prevents growth of cells, e.g. HSV-TK. In step 2, the RNA is fastened to the gene transfer material (e.g. to liposoms). In step 3, the RNA bearer mixture is injected to the inner wall of the blood vessel in connection with balloon extension. In step 4, the RNA is expressed in the cells of the blood vessel walls, is replicated and forms VL-particles. In step 5, the particles are spread to the cells of the target area and the transfer gene is expressed. The growth of the cells stop and the forming of a new blockage is prevented.

The synthesis of RNA takes in all embodiments place by using a starting plasmid taken from a Sindbis or Semliki forest virus. The first mentioned plasmid is e.g. a commercial product which is marked PSINREP5, from which the recombinant plasmid of the invention is made, the second mentioned plasmid is PSFVDP6-X, from which the recombinant plasmid is made in accordance with the ideas of the article (Rolls et al, 1994, Cell 79, 497-506). The ring of the starting plasmid is cut in the synthesis, i.e. linearized and SP6 RNA-polymerase is used for the synthesis of plus-stranded RNA, i.e. the replicon.

In FIGS. 7 and 8, the RNA is fastened to the bearer particles with a known method. The bearer particles are commercial carrier gold particles in other words bearer gold particles provided from e.g. the Biorad company. In the method, the fastening takes place for example by means of ethanol precipitation, whereby the nucleic acids are precipitated with the gold particles, adhere to the precipitate, which sinks to the bottom of the vessel. The ethanol is then evaporated and the RNA stays on the surface of the gold particles.

In the gene gun method, there is for example used particles coated with Sin-G-dp-TKGFP-RNA in the invention, which thus are particles to which the RNA fastened to them forms VL particles of Sin-G-dp-TKGFP on the cells. The particles are placed on the inner surface of a plastic hose. The hose is placed into a device (gene gun) and a strong gas flow is lead from the helium gas bottle to the hose, which leads the particles from the inner surface of the hose to the target area.

In the following some embodiment examples are presented of the functionality of the invention.

### EXAMPLE 1

Transfer of RNA to BHK Cells with Electroporation, Spreading of VL Particles in BHK Cells

In this example it is shown that by transferring VLP RNA to BHK cells by means of electroporation, VL particles are formed, which spread to new cells and increase the amount of cells expressing the transfer gene. It is also shown that be spreading of the VL particles is dependent on be cell density and require an active dividing of the cells to take place.

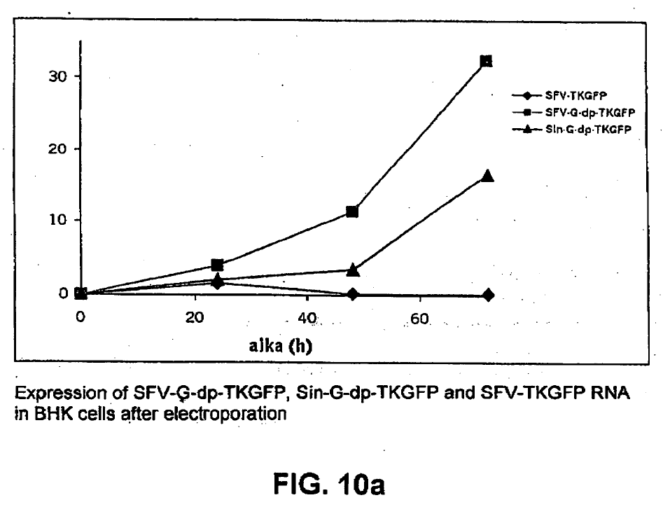

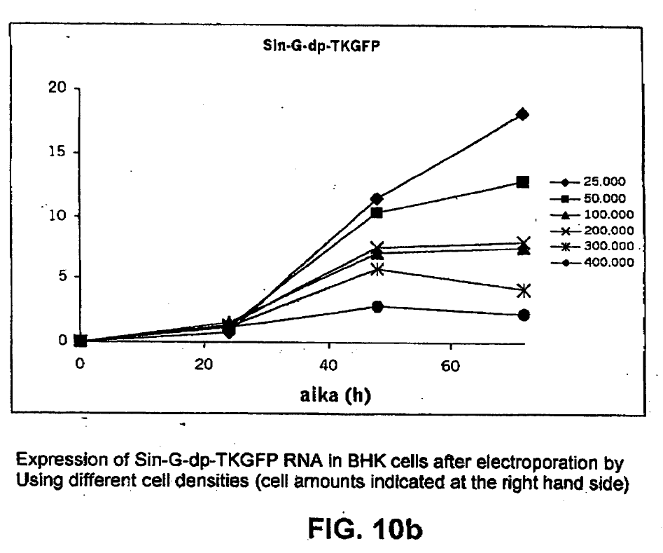

In the first test, pSFV-, TKGFP-, pSFV-G-dp-TKGFP or pSin-G-dp-TKGFP RNA was transferred to the BHK cells by means of electroporation. The part of the positive cells in the culture as followed by means of flow cytometry and fluerescence microscopy during 72 hours. pSFV-TKGFP RNA was used as control and it encodes the conventional SFV vector, to which a TKGFP gene has been connected. This RNA can not form VL particles when being transferred to the cells or spread to new cells. This can clearly be observed in FIG. 10a, wherein the SFV-TKGFP positive BHK cells disappear from the culture in 2 days. On the contrary, those cells to which VL particles encoding RNA has been transferred (pSFV-G-dp-TKGFP or pSin-G-dp-TKGFP) begin to produce spreading particles, whereby the amount of TKGFP positive cells in the culture grow in time.

In the second step, the effect of the cell density on the spreading was studied. The cells can divide only when the incubating vessel has not grown full so in this way the effect of the cell dividing activity on the spreading of the VL particles can be tested.

Sin-G-dp-TKGFP RNA was electroporated to the BHK cells and these cells were divided to incubation dishes in different amounts in such a way that in the dish that contained the least amount of cells (25,000 cells) the dividing of cells took place during the whole investigation period, whereas the dish that contained the biggest amount of cells (400,000 cells) was grown full already during the first day and the cell dividing had stopped completely. From FIG. 10b it can be seen that the dividing of the VL particles is completely dependent on the dividing of the cells and when the cells reach the maximal density (and the dividing has stopped) also the increase of amount of the TGKFP positive cells stops.

The results of these tests show that SFV-G-dp-TKGFP or Sin-G-dp-TKGFP RNA forms such VL particles when transported to the BHK cells that can spread to the other cells of the culture. The spreading, however, only continues as long as the cells are dividing. This can be considered to be a positive property as for example the spreading in gene therapy tests for cancer can been assumed to be restricted to dividing cells, which is what most of the malicious cells are like.

### EXAMPLE 2

Transfer of RNA to Other Cells than BHK Cells by Electroporation, Spreading of VL Particles

In this example it is shown that VL particles prepared with the method can be formed and the expression of the transfer gene maintained also in other cells than BHK cells.

In this test mouse oligodendrocyte cell line MBA13 was used, pSFV-G-dp-TKGFP or pSin-G-dp-TKGFP RNA were transferred to the cells by means of electroporation. The part of the positive cells in the culture was followed by means of flow cytometry and fluorescence microscopy during 52 hours.

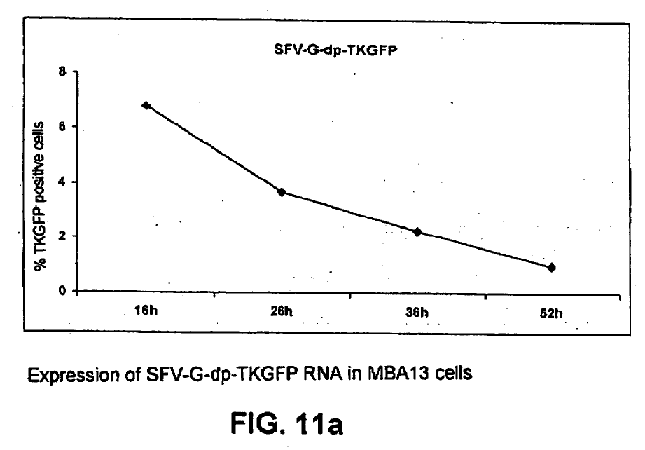

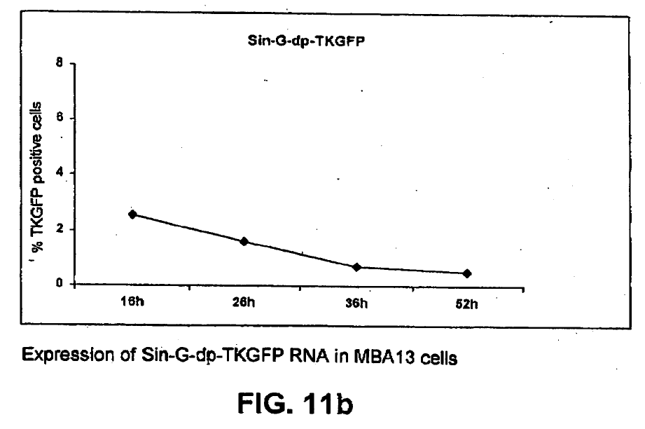

In can be seen from FIGS. 11a and 11b (the part of positive cells defined with flow cytometry) that in the cells in question any explosive growth does not take place in the amount of positive cells, instead a little decrease. The decrease is, however, clearly slower when using conventional SFV-TKGFP RNA, whereby the amount of positive cells has a top after 24 hour electroporation and goes down to zero level during the following day (see FIG. 10a).

The results of these tests show that the function of VL particles formed by the RNA transferred by electroporation the functioning of VL particles in the MBA12 cells are different than in BHK cells. It has to be assumed that the explosive spreading of the particles only takes in the BHK cells and in other cell types the amount of the cells can be observed to stay on the same level or to decrease slowly. This is a positive properly compared to conventional SFV or Sin—vectors in which the positive cells disappear (die) very quickly.

It also has to be noted that no destroying of cells where observed in the MBA13 cells whereas in the BHK cells also dying of cells could be observed along with the spreading of the VL particles. This makes it possible to use the method also in situations, herein a destroy of target tissue is not recommendable (e.g. in vaccine use).

### EXAMPLE 3

Use of the Method of the Invention in Gene Therapy of Cancer

In this example it is shown that the VL particulles can be used as gene transfer vectors in gene therapy of cancer.

It the gene therapy method to be used a gene of virus origin (HSV-TK) changes the virus medicine that is harmless for human beings (ganciclovir GCV) to a toxic form. When the HSV-TK gene is transferred to the cell it changes to be sensitive for the GCV- medicine and is destroyed even with small amounts of the medicine. Both the VLP-constructs described in this application (SFV- and Sin-G-dp-TKGFP) contain the HSV-TK- gene as a part of the TKGFP fusion gene so they can be used as such in the gene therapy tests.

In the test, VLP-RNA was transferred by means of electroporation to the MBA13 cells. The part of the VLP positive cells was determined with flow cytometry. Normal cells were mixed with these calls in such way that a cell population was achieved in which the part of VLP positive cells was in the range of 0-10%. 10 mg/ml of these cell populations mere incubated in the presence of GCV in 5 days where after the life ability of the cells was determined with so called MTT-analysis (where the amount of the color to be formed and thus the absorbance in the spectrophotometric measurement is directly proportional to the life ability of the cells).

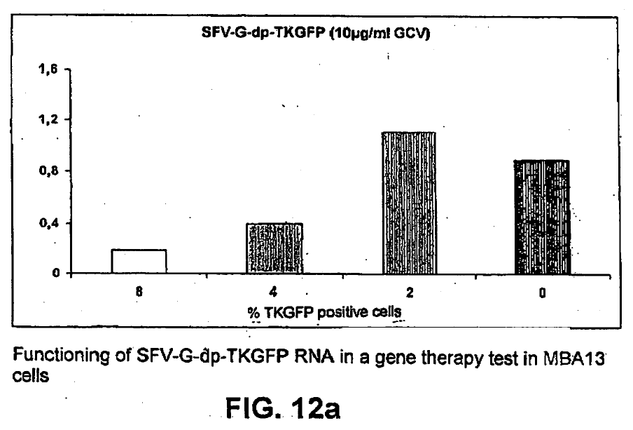

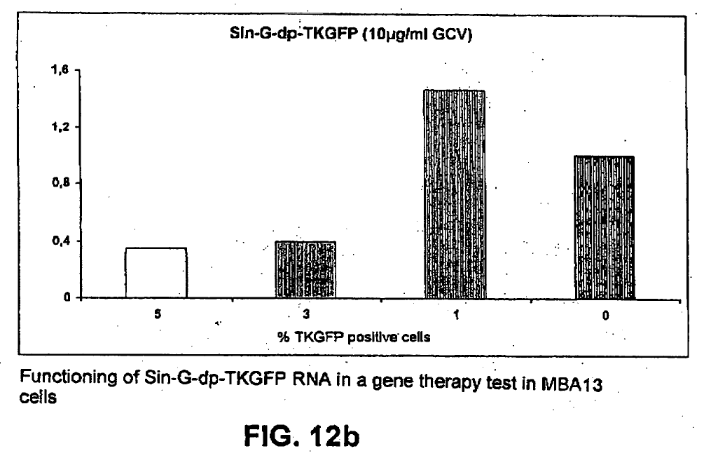

As is observed in FIGS. 12a and 12b, only 3-4% of positive cells is sufficient to make the whole cell population ca three times more sensitive to the GCV-medicine. This is a very good result and a corresponding effect is achieved with the use of conventional virus vectors only when the population contains at least 10-14% positive cells.

### EXAMPLE 4

Transfer of RNA to BHK-Cells with the Gene Gun Method, Proving Functionality In Vitro and In Vivo

In this example it is shown that VLP-RNA (here pSin-G-dp-TKGFP) can be bound to the gold particles to be used in the gene gun technology and be transported in a functional form to the target cells.

RNA is fastened to gold particles that are delivered by the manufacturer (BioRad) to the inner surface of a silicon tube by means of ethanol precipitation in accordance with the instructions of the manufacturer. A hose coated with RNA gold particles we cut to suitable pieces (ca. 1 cm) and is loaded to the gene gun device (Helios, BioRad). The device is combined with a helium gas container and the particles are shot by means of a gas impulse to the target cells,

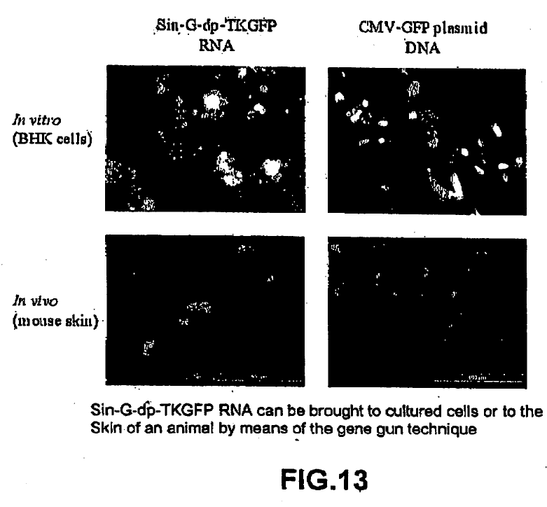

In the first step, cultured BHK-cells were used (the upper row of FIG. 13) and in the second step there was used stomach skin of a nude-mouse (lower row of FIG. 13). From the figure it can be observed that both in the cultured cells and in the living animal the VLP-RNA, transferred with the gene gun method, works normally and a good transfer efficiency is achieved. Furthermore it can be stated that by using VLP-RNA much cells can be observed in which the expression level of the transfer is clearly higher compared to the control (instead of RNA the plasmid DNA=CMV-GFP was connected to the particles).

1. Method of preparing of treatment product characterized by
a) using a starting plasmid based on a virus belonging to the togaviridae family, from which the gene encoding the capsid protein of the virus has been removed,
b) virus-like particles encoding RNA (VLP-RNA) is prepared by changing said starting plasmid by connecting to it a gene enabling spreading and a treatment gene.

2. Method of claim 1 characterized in that the starting plasmid based on the virus belonging the togaviridae-stock is based on an alphavirus, especially the Sindbis or Semiliki forest virus.

3. Method of claim 2 characterized in that the starting plasmid to be changed is the pSFVdpG-X-plasmid of FIG. 1 of the Semliki forest virus or the pSinRep5-plasmide of FIG. 3 of the Sindbis-virus.

4. Method of claim 3 characterized in that the pSFV-G-dp-TKGFP-pasmid construct respective the pSin-G-dp-TKGFP-plasmide construct is prepared from the starting plasmids of the foregoing patent claim.

5. Method of claim 4 characterized in that the pSFV-G-dp-TKGFP-plasmid construct is prepared by opening the pSFVdpG-X-plasmid with a restriction enzyme and by connecting a treatment gene TGKFP to the opened plasmid.

6. Method of claim 4 characterized in that the pSinG-dp-TKGFP-plasmid construct is prepared by opening the pSinRep5-plasmid with a restriction enzyme and by connecting VSV-G or TKGFP to the opened plasmid.

7. Method of claim 6 characterized in that the pSinG-dp-TKGFP-plasmid is prepared from the pSinRep5-plasmid by
connecting the VSV-G gene to the plasmid,
by connecting another subgenomic promoter to the plasmid,
by connecting the TKGFP-gene behind the foregoing one,
isolating the plasmid pSin-G-dp-TKGFP and
by massgrowing the plasmid for RNA synthesis.

8. Method of any of claims 1-7, characterized in that a solution for gene therapy of cancer is prepared by using, as a treatment gene, a gene that is toxic for cancer cells and/or a gene that increase, the immune response.

9. Method of claim 8 characterized in that the treatment gene used is the thymidine kinase gene HSV-TK or herpes simplex virus type 1.

10. Method of any of claims 1-7, characterized in that a solution intended for vaccine use is prepared by using a vaccine gene and/or a gene that increases the immune response as a treatment gene.

11. Method of claim 10 characterized in that the treatment gene is a surface protein gene of virus p21 of HIV1 or interleukin12-gene.

12. Method of any of claims 1-7, characterized in that a solution meant for preventing re-growth of blood vessels is prepared by using a gene that prevents growth of cells as a treatment gene.

13. Method of claim 12 characterized in that HSV-TK is used as a treatment gene.

14. Treatment product characterized in that it is a virus-like particles encoding RNA (VLP-RNA) that contains a gene that enables spreading and a treatment gene.

15. Treatment product of claim 14 characterized in that the gene that enables spreading is VSV-G.

16. Treatment product of claim 14 characterized in that the treatment gene is TKGFP.

17. Plasmid construct encoding for virus-like particles, characterized in that, it is a construct prepared from the Sindbis virus, wherein the capsid protein of the virus has been substituted by a gene that enables spreading and by a treatment gene.

18. Construct of claim 17, characterized in that, the gene that enables spreading VSV-G.

19. Construct of claim 17, characterized in that, the treatment gene is TGKFP.

20. Construct of any of claims 17-19, characterized in that, it is pSIN-G-dp TKGFP according to FIG. 4.
